(19) United States
(12) Patent Application Publication
(10) Pub. No.: US 2025/0382655 A1
(43) Pub. Date: Dec. 18, 2025

(54) MULTI-PARAMETRIC METHOD FOR IDENTIFICATION, QUANTIFICATION AND IN-VIVO ESPONSE ASSESSMENT OF VIABLE MICROBIAL ORGANISM FROM BIOLOGICAL SPECIMENS

(71) Applicant: MedhAnukura Private Limited, Hyderabad (IN)

(72) Inventors: Attili Venkata Satya Suresh, Hyderabad (IN); Peesa Jagdeesh, Hyderabad (IN); Vutukuru Anuradha, Hyderabad (IN)

(21) Appl. No.: 18/878,241

(22) PCT Filed: Feb. 26, 2023
(86) PCT No.: PCT/IB2023/051770

§ 371 Date: Dec. 23, 2024

(30) Foreign Application Priority Data
Sep. 10, 2022 (IN) 202241051771

(51) Int. Cl. C12Q 1/06; C12Q 1/18; C12Q 1/48; C12Q 1/6851; C12Q 1/686

(52) U.S. Cl. CPC C12Q 1/06; C12Q 1/18; C12Q 1/48; C12Q 1/6851; C12Q 1/686; C12Y 207/07

(57) ABSTRACT

A multi-parametric method for identification, quantification and in-vivo response assessment of viable microbial organism from biological specimens. The proposed method uses multiple parameters for identification of viable microbial organisms from biological specimens. The proposed method utilizes live media for the first few cycles of bacterial growth in the biological sample. The proposed method determines the microbial burden based on the biological doubling time of a microbe as determined by the nomogram. The proposed method performs growth assessment based on Chromomeric dissociation, impedance matching, pH assay and turbidity assay, during the transport of the biological sample. The proposed method avoids measuring dead pathogens/microbes for effective assessment of the biological sample to determine the dose, frequency and nature of the antibiotics that are appropriate to achieve control of microbial infection.

### FIELD OF THE INVENTION

The present disclosure generally relates to the technical field of study of biological specimen culture, and in specific relates to a method for detection of presence of viable microbial organism through identification of multiple parameters of the viable microbial organism from a biological specimen and derive the response assessment in-vivo using array of artificial intelligence techniques.

### BACKGROUND

In general, when treating a newly diagnosed sick patient, healthcare professionals, those who specialize in antimicrobial treatment, find it difficult to select the correct antimicrobial medicines. In microbial diagnostics, laboratory testing of clinical specimens is important for the presence of microbial species, pathogens and bacteria. In traditional methods for microbial diagnosis, a sample of clinical specimens from the suspected infection, such as blood, urine, cerebrospinal fluid, and semen thereof, is collected and sent for microbial diagnosis. The collected clinical specimens are submitted for the identification and isolation of the organism. Once the specimen reaches the laboratory, the specimen is grown in culture media, which typically takes few days to weeks. During the growth of the specimen, the healthcare professionals keep checking for the appropriate antimicrobial agents, which either kill or block the growth of the specimen.

However, it is quite difficult to standardize microbial growth methods and content requirement of the culture media, as each of the microbial species or pathogen need particular requirements. Further, the traditional methods are also time-consuming, as microorganisms normally grow in solid culture media or liquid broths for 24 to 48 hours (or longer in the case of slow-growing organisms like Mycobacterium tuberculosis). After the identification of an organism, antimicrobials are determined that can either kill the organism or stop its development. This is normally done on culture media and is frequently reported as MIC/Zone of Inhibitions, which may take an equivalent amount of time.

At present, various methods are used for fast microbial identification such as DNA sequencing to identify bacteria, moulds and yeasts, riboprinter analysis for bacterial identification and characterization, and polymerase chain reaction for assessing the similarity of microorganisms. Rapid detection of bacteria is important for the diagnosis process. Modern technologies such as microarray, Raman spectroscopy, mass spectrometry, DNA sequencing as well as multiplex real-time PCR have become the most used tools within this field, which accelerate the development of the health industry.

In microbial diagnosis, differentiation of live and dead cells is a significant difficulty. In the case of pathogenic microorganisms, the potential health risks are limited to the live portion of a mixed microbial population. Using fluorescent stains, flow cytometry can discriminate between four physiological states of microbes such as reproductive viability, metabolic activity, intact cells and permeabilized cells. All stages, except the permeabilized cells, are regarded as potentially alive because they may recover upon resuscitation, depending on the circumstances. However, DNA-based diagnostics tend to overestimate the number of live cells because they also measure the DNA from dead cells. DNA extracted from a sample originates from cells in any of the four above mentioned physiological states including the dead permeabilized cells. Therefore, DNAbased diagnostics cannot distinguish between live and dead bacteria. Polymerase chain reaction (“PCR”) is used as a tool for the quick detection of bio-threat and foodborne pathogens. However, PCR itself does not discriminate from DNA coming from live pathogens (harmful) or dead pathogens (harmless).

However, the existing rapid identification technologies of microbes fail to differentiate between living microbes and dead microbes and thereby affect the calculation of bacteria growth in biological specimens. The existing rapid identification technologies are not capable of detecting viable microbial organisms from all biological specimens such as blood, solid tissue and CSF thereof. The existing technology is unable to perform quantitative monitoring of the response to therapy, which limits the healthcare professionals to instantly change the antimicrobe for better clinical outcomes.

Therefore, there is a need for a method that detects the presence of viable microbial organisms through the identification of multiple parameters of a viable microbial organism from biological specimens in a rapid method. There is a need for a method that differentiates between living microbes and dead microbes for calculation of bacteria growth in biological specimens. A responsive method is needed for quantitative monitoring of the response to antibiotics. There is a need for a method that aids healthcare professionals to instantly change the anti-microbe or antibiotics for better clinical outcomes. A method is needed that is capable of detecting viable microbial organisms from any biologic specimens.

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key nor critical elements of all embodiments, nor delineate the scope of any or all embodiments.

The present disclosure, in one or more embodiments, relates to a multi-parametric method for identification, quantification and in-vivo response assessment of viable microbial organism from biological specimens. Furthermore, the Multi-Parametric Method for Identification, Quantification and In-vivo Response Assessment of Viable Microbial Organism from Biological Specimens. First, a primary specimen is collected in a broth culture for natural growth of microbes to obtain a microbes broth. The time for collection of the primary specimen is recorded. In specific, the primary specimen includes a biological sample such as either blood or solid tissue or any biological sample. Next, the microbes broth is subjected through plurality of growth assessments to determine a first microbial burden in the microbes broth. In specific, the growth assessments include colorimetric assay, pH assay and impedance matching of the microbes broth.

Next, biological doubling time of a microbe is determined through nomogram. Next, the microbes broth is subjected for sub-culturing. In specific, the microbes broth is placed in a live media to obtain a subculture specimen. Then, minimum inhibitory concentration (MIC) of the subculture specimen is determined. In specific, the live media includes soybean casein agar that allows natural growth of the gram positive, gram negative bacteria. The sub-culturing of the microbes broth is preformed to trap dead microbes on the live media.

Next, the subculture specimen is subjected through a standard quantitative polymerase chain reaction (PCR) to obtain an amplified specimen. In specific, correction factor is utilized during the PCR. Next, the amplified specimen is analysed for the potential resistant genes to determine an antibiotic. Further, the amplified specimen is augmented with conventional automated culture technique to determine the MIC concentrations.

Next, the determined antibiotic is administrated with the amplified specimen to obtain a secondary specimen. Next, the secondary specimen is placed in the live media to determine a second microbial burden after a defined time. Later, difference between the first microbial burden and the second microbial burden is calculated to obtain the growth and type of the microbes. Finally, a response assessment score is calculated based on the growth of microbes through an artificial intelligence module. In specific, the response assessment score is utilised to determine dose, frequency and nature of the antibiotics that are appropriate to achieve control of microbial infection.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates an exemplary Multi-Parametric Method for Identification, Quantification and In-vivo Response Assessment of Viable Microbial Organism from Biological Specimens in accordance to an exemplary embodiment of the invention.

FIG. 2 illustrates an exemplary representation of timed automated spill for the secondary cultures in accordance to an exemplary embodiment of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

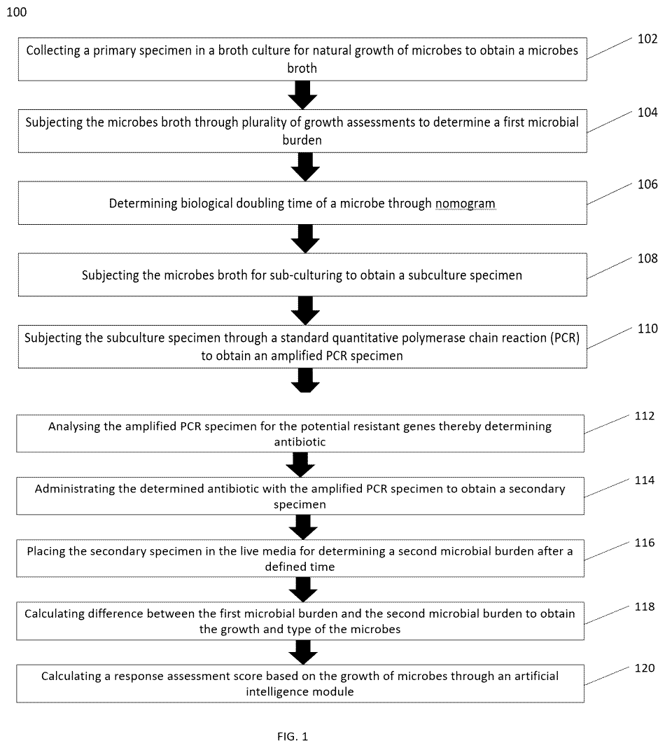

FIG. 1 refers to an exemplary multi-parametric method 100 for identification of viable microbial organism from biological specimens. At step 102, a primary specimen is collected in a broth culture for natural growth of microbes to obtain a microbes broth. The time for collection of the primary specimen is recorded. In specific, the primary specimen includes a biological sample such as either blood or solid tissue or any biological sample collected from a patient.

For instance, soybean casein agar is taken, but not limited to an initial transport media to collect the primary specimen. This unique combination of the ingredients in the soybean casein agar allows the natural growth of the gram positive, gram negative bacteria. The other microbial species remain static on this media. Thereby, transport time is utilised, for the first few cycles of the bacterial growth of the microbes.

At step 104, the microbes broth is subjected through plurality of growth assessments to determine a first microbial burden in the microbes broth at the end of a finite time. In specific, the growth assessments include colorimetric assay, pH based assay and impedance matching of the microbes broth.

In the Colorimetric/Chromometric assay, the bacterial cell count in the primary specimen is monitored using the turbidity. In specific, a fixed quantity of light is passed through the microbes broth, the opacity is directly proportionate to the growth, which are derived from the nomograms. Further, in another method, salts are mixed in the microbes broth. Due to the addition of salts change in the colour of the microbes broth is observed. In specific, the salts aid in the growth of bacteria and provide to change in pH and other metabolites.

For instance, tetrazolium salt also known as MTT [3-(4,S-dimethylthiazol-2-yl}-2,5-diphenyltetrazolium bromide) is used. The measurement is based on the spectroscopic analysis of the microbes broth.

In pH based assay, the production of the carbon dioxide by the growing bacteria, which interacts with the moistures, is observed to drop the pH. The pH is measured using pH probes and nomograms. The nomograms are used for deducting the bacterial growth in the microbes broth. In Impedance matching, the change in the electrical activity of the microbes broth as well as impedance of the growing bacterial colony is measured by the probes. Further, the mapped is performed on nomograms to deduce the bacterial growth. Further, after the growth assessments the first microbial burden is determined, which give a primary estimate of the bacterial burden at baseline and at the end of the finite time.

At step 106, biological doubling time of a microbe is determined through nomogram. In specific, a graph for the microbial burden is determined in a finite time. From the collection of the primary specimen to the analysis by PCR forms the basis for microbial burden. For instance, if a microbe doubles in 10 minutes and the primary specimen is analysed after 1 hour. Then, the possible cycles of growth as per the nomogram are determined and use the correction factor during the PCR measurement.

At step 108, the microbes broth is subjected for sub-culturing. In specific, the microbes broth is placed in a live media to obtain a subculture specimen. Then, minimum inhibitory concentration (MIC) of the subculture specimen is determined. In specific, the live media includes soybean casein agar that allows natural growth of the gram positive, gram negative bacteria. The sub-culturing of the microbes broth is preformed to trap dead microbes on the live media. The soybean casein agar disclosed in the embodiment is not confined only for soybean casein agar any other live media that provides natural growth of the gram positive, gram negative bacteria can also be considered.

In the embodiment, the aghar is preparation, by suspending 45 grams (range 20-55) of Soyabean Casein Digest Agar in 900 ml (range 700-1200) of distilled water. The soyabean casein digest agar consists of casein enzymic hydrolysate 15.00 grams, papaic digest of soya bean meal 5.00 grams, sodium chloride 5.00 grams, agar 15.00 grams.

At step 110, the subculture specimen is subjected through a standard quantitative polymerase chain reaction (PCR) to obtain an amplified specimen. In specific, the subculture specimen is subjected to a standard quantitative PCR method for the genetic amplification and matched with a standard library to confirm the microbe of interest. In parallel to quantification of microbial burden, the correction factor for the transport media time is taken into account for the final calculations of microbial burden, which is deducted using standard regression model to give the CFU/Colony forming units. In specific, the transport media time is calculated from the collection of the primary specimen till the amplified specimen is obtained. Further, correction factor is utilised during the PCR. The standard quantitative polymerase chain reaction (PCR) disclosed in the embodiment is not confined only for PCR it can be any other similar quantitative methods.

At step 112, the amplified specimen is analysed for the potential resistant genes to determine an antibiotic. Further, the amplified specimen is augmented with conventional automated culture technique to determine the MIC concentrations. In specific, the viable microbial organism is identified from the amplified specimen. Thereby the antimicrobial agent (antibiotic) is determined. At step 114, the determined antibiotic is administrated with the amplified specimen to obtain a secondary specimen. At step 116, the secondary specimen is placed in the live media to determine a second microbial burden after a defined time. In specific, the decrement or increment in the microbial burden is determined after the defined time.

At step 118, difference between the first microbial burden and the second microbial burden is calculated to obtain the growth and type of the microbes. At step 120, a response assessment score is calculated based on the growth of microbes through an artificial intelligence module. In specific, the response assessment score is utilised to determine dose, frequency and nature of the antibiotics that are appropriate to achieve control of microbial infection.

For instance, a microbe can be sensitive to an antimicrobial agent, but once it enters body an array of biological and micro environmental factors either enhance or reduce the antibiotic effect. Hence, the proposed method measures the actual response using a score to determine dose, frequency and nature of the antibiotics that are appropriate to achieve control of microbial infection.

For instance, Table 1 depicts a normogram for microbial burden detection of a pathogen or microbe E. coli.

TABLE 1

| Y/X | 10² | 10³ | 10⁴ | 10⁵ | 10⁶ | 10⁷ |
|---|---|---|---|---|---|---|
| 10 min | 10³ | 10^{3.2} | 10^{3.8} | 10^{4.2} | 10^{5.0} | 10^{5.2} |
| 20 min | 10^{2.5} | 10³ | 10^{3.2} | 10^{3.8} | 10^{4.5} | 10^{5.0} |
| 30 min | 10^{2.2} | 10^{2.5} | 10³ | 10^{3.2} | 10^{3.8} | 10^{4.0} |
| 40 min | 10^{2.0} | 10^{2.2} | 10^{2.5} | 10^{2.5} | 10^{3.4} | 10^{3.8} |
| 50 min | 10^{1.8} | 10^{2.0} | 10^{2.2} | 10^{2.3} | 10^{2.5} | 10^{3.6} |
| 60 min | 10^{1.6} | 10^{1.8} | 10^{2.0} | 10^{2.2} | 10^{2.2} | 10^{2.0} |

Wherein X represent observed bacterial count and Y represent time at which the bacterial count is taken. For example, if at 60 minutes the bacterial count is 106, then at the starting time the actual bacterial count was 102.2. The actual bacterial count is deducted by mapping time with observed bacterial count.

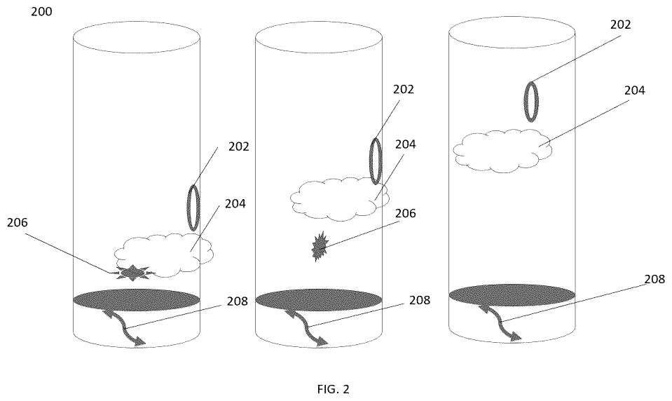

FIG. 2 refers to an exemplary representation 200 of timed automated spill for the secondary culture. A spill over 202 is configured to capture secondary cultures from the subculture specimen 204 (microbes broth). The spill over 202 is placed at fixed height, so that secondary cultures are captured without contamination. A spring 208 is configured with timers to enable the subculture specimen 204 to spills to next culture. During this process, only live microbes are taken and the culture media exposure is provided to microbe 206 for a definite time.

According to another exemplary embodiment, the invention utilises a method for quantitative microbial response assessment. A sample is collected from the patient at different points of time and change in the microbial burden (increase/decrease) is observed. Any change is quantified using the artificial intelligence module based response assessment score to aid clinicians determine dose, frequency and nature of the antimicrobial agent. The response assessment score aids the clinicians to make a decision on either to continue or to alter the antimicrobial agent based on improvement of the patient.

According to another exemplary embodiment, the invention utilises an artificial intelligence module for deduction of the response assessment score. First, viable microbial organism is identified. The MIC values from the conventional sensitivity test, gene expression of resistant genes and accelerated sensitivity test are correlated with each other. If, the MIC values are concurrent, then a response assessment score is generated by the artificial intelligence module. Else if, the MIC values are not concurrent, then the artificial intelligence module is subjected for training. The artificial intelligence algorithm is trained with dataset of various microbial organisms and their response to provide accurate MIC values.

In specific, a first microbial dataset is subjected for training and validation. The first microbial dataset is formed from actual microbial burden measured by gold standard and the response of a patient towards the antimicrobial agent, the anti-microbial sensitivity using either a standard technique or PCR based technique. Then, scoring for the first microbial dataset is performed, in which the rule such as either k-nearest neighbours algorithm (KNN) or decision tree or Artificial neural networks (ANN) or Convolutional Neural Network (CNN) is used to derive a machine learning model. Then, a new microbial dataset of a new patient, returns outcomes in the form of probability scores for classification problems and estimated averages for regression problems. In specific, the machine learning model provides scores by applying an algorithmic model built from a historical dataset (first microbial dataset) to a new dataset (new microbial dataset) in order to obtain practical observations.

For example, in the first microbial dataset a patient X responded either better or worse than the lab suggested drug, then these observations are utilised for building the machine learning model and derive an accurate score.

The machine learning models constantly undergo supervised learning for the deployment of a trained model into a production application. Further, use the observations for determination of correct antimicrobial or combination antimicrobial which provide the fast and best the result in a patient. When the response assessment score is low, the patient's recovery rate is high due to less microbial burden. When the response assessment score is high, the patient's recovery rate is low due to high microbial burden.

According to another exemplary embodiment, the invention utilises multivariate imputation method for the microbial burden. In case, the growth assessments, which include colorimetric assay, turbidity assay, pH based assay and impedance matching, are not completed or any data of the growth assessments is not available (missed data). Then, a dataset of available feature dimensions is used to estimate the missing data. The missing data includes cycles of the bacterial growth such as the bacterial growth at a specific time, or the microbial burden at the specific time thereof. In specific, the missing data are filled in to create a complete dataset. Thereby the complete dataset is analysed using standard methods.

For example, during turbidimetry assay of the primary specimen, a section of time is not recorded. In this case the missing data is the section of time, which affects the bacterial cell count in the primary specimen. Then, based on the similarly data from the existing dataset for that particular section of time, specific to the microbe is filled in to create a complete dataset. In specific, turbidimetry is imputed and the similarly data is added in the calculation of the microbial burden.

At each step of the multivariate imputation methods, a feature column is designated as output y and the other feature columns are treated as inputs X. A regressor is fit on (X, y) for known y. Then, the regressor is used to predict the missing data of y. This is done for each feature in an iterative manner, and then is repeated for multiple imputation rounds. The results of the final imputation round are used for response assessment score.

For instance, one miss-match, PCR based gene is added with MIC values, which is supplemented with a corrected accelerated score. MIC values are added with accelerated match, which is supplemented with a corrected PCR score. PCR based gene is added with accelerated match, which is supplemented with a corrected MIC score. For two miss-matches, repeat the fastest growth assessments to obtain 90% to 110% range. If the range is not achieved, then repeat the growth assessments to obtain 85% to 120% range. If still the range is not achieved, then reject the sample as an error.

In case, more than one data are missing. Then, a k-Nearest Neighbours technique is used to provide imputation for filling in the missing data. In specific, a KNNImputer class is used to provide imputation for filling in missing values. First, a euclidean distance metric (nan_euclidean_distances), that supports missing values, is used to find the nearest neighbors of the missing data. Then, each missing data is imputed using values from n_neighbors, the n_neighbors are the nearest neighbors that have a value for the missing data.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A multi-parametric method for identification, quantification and in-vivo response assessment of viable microbial organism from a biological specimen derived from a living organism, comprising:
collecting a primary specimen in a broth culture for natural growth of microbes to obtain a microbes broth, thereby recoding time of collection of said primary specimen;
eliminating the supernatant to avoid assessment/analysis of the dead microbes and allowing the secondary culture only to determine the further characterization of live microbes alone for subsequent analysis;
subjecting said microbes broth through plurality of growth assessments to determine a first microbial burden in said microbes broth;
determining biological doubling time of a microbe through nomogram;
subjecting said microbes broth for sub-culturing by placing said microbes broth in a live media to obtain a subculture specimen and determining minimum inhibitory concentration (MIC) of said subculture specimen;
In parallel subjecting said subculture specimen through a rapid standard quantitative methods to obtain an amplified specimen and matching with library to obtain the classification of the microbe (like gram positive/negative, species sub species etc) to give a rapid report within time much earlier than the conventional culture, where said standard quantitative method is similar to polymerase chain reaction (PCR) method;
analysing said amplified specimen for the potential resistant genes, thereby determining an appropriate antibiotic and suggesting for the use in the person for the control of the microbial growth;
further augmenting said amplified specimen with conventional automated culture technique for determining the MIC concentrations, comparing and sending the said data for the supervised learning models of AI/ML (Artificial Intelligence/Machine learning);
after administrating said determined antibiotic in the Source/person (from where the specimen is obtained), to verify the clinical response and get serial samples/specimens to monitor the microbial growth as well as response assessment via quantitative assessment and sending he data back for the supervised learning models of AI/ML (Artificial Intelligence/Machine learning) to assist the score;
placing said subsequent specimen in a live media for determining a subsequent microbial burden after a defined time;
calculating difference between said first microbial burden and said subsequent microbial burden to obtain the growth and type of the microbes; and
calculating a response assessment score based on said growth of microbes through an artificial intelligence module,
whereby said method detects the presence of viable microbial organisms through the identification of multiple parameters of a viable microbial organism from the biological specimen.

2. The multi-parametric method of claim 1, wherein said plurality of growth assessments include conventional visual assesseemtnon plaate, colorimetric assay, pH assay, and impedance matching of said microbes broth.

3. The multi-parametric method of claim 1, wherein said live media includes soybean casein agar that allows natural growth of the gram positive, gram negative bacteria.

4. The multi-parametric method of claim 1, wherein said sub-culturing of said microbes broth is preformed to trap and eliminate dead microbes on said live media.

5. The multi-parametric method of claim 1, wherein utilising said response assessment score for determining dose, frequency and nature of said antibiotics that are appropriate to achieve control of microbial infection.

6. The multi-parametric method of claim 1, wherein primary specimen includes a biological sample such as either blood or solid tissue or any biological sample.

7. The multi-parametric method of claim 1, wherein a correction factor is utilised during the PCR method.

8. The multi-parametric method of claim 1, wherein the artificial intelligence module is trained with dataset of various microbial organisms and their response to provide accurate MIC concentrations values.

9. The multi-parametric method of claim 1, wherein the nomograms are used to determine bacterial growth in the microbe broth and map time to deduce the bacterial growth.
